(19) United States
(12) Patent Application Publication
(10) Pub. No.: US 2008/0071340 A1
(43) Pub. Date: Mar. 20, 2008

(54) Implantable electrodes with polyoxometalates

(75) Inventors: Liliana L. Atanasoska, Edina, MN (US); Jan Weber, Maastricht (NL); Roger N. Hastings, Maple Grove, MN (US); Robert W. Warner, Woodbury, MN (US); Jeannette C. Polkinghorne, St. Anthony, MN (US)

Correspondence Address: Brooks & Cameron, PLLC, Suite 500, 1221 Nicollet Avenue, Minneapolis, MN, 55403, US

(73) Assignee: Boston Scientific Scimed, Inc.

(21) Appl. No.: 11/521,966

(22) Filed: Sep. 15, 2006

(51) Int. Cl. A61N 1/00

(52) U.S. Cl. 607/121

(57) ABSTRACT

An electrode with an electrode surface having a polyoxometalate (POM). The use of POM with an electrode surface increases the active electrochemical surface area, with a resulting increase in capacitance and impedance, and a decrease of polarization losses at the electrode/tissue interface. In addition, electrodes having POM can include pseudo-capacitive properties from their redox properties and charge storage properties.

### TECHNICAL FIELD

The present disclosure relates to biomaterials containing polyoxometalate (POM) structures. More particularly, the disclosure relates to implantable electrodes having POM structures.

### BACKGROUND

Implantable electrodes for electrical stimulation and sensing can be quite small. One driving force for the reduction in electrode size is the increase in possible locations for implanting the electrode. In addition, the smaller electrode size also can lower stimulation thresholds and increase power supply (e.g. battery) longevity. As can be appreciated, extending battery life allows for a longer potential service life of the implanted device (e.g., pacemaker). However, with reduction of the size of the electrode (e.g., a reduction in the geometric surface area of the electrode) there is an increase in current density across the electrode. This increase in current density can increase the possibility to exceed safe electrical charge limits, which could result in electrode material dissolution, electrolyte redox reactions, and/or the production of toxic chemicals.

In an effort to control the current density various options have been suggested and used to increase the actual electrode surface area without increasing the overall physical dimensions of the electrode. Examples of such options include porous electrode materials, sintered microspheres, fractal electrode surface morphology, and fractally coated electrodes. There, however, continues to be a need for large actual electrode surface area while not increasing the overall physical dimensions of the electrode.

### SUMMARY

Embodiments of the present disclosure provide for implantable electrodes that include polyoxometalate (POM). In the various embodiments, the POM may provide the implantable electrode with an electrochemically active and flexible low polarization pseudo-capacitive electrode surface. Electrode surfaces that include POM may be suitable for delivering low to high voltage stimulation pulses, for example up to 10 volts, without exceeding a safe charge injection limit and electrochemical potential window. In addition, implantable electrodes that include POM may also display reduced polarization losses at the electrode/tissue interface.

As used herein, “polyoxometalate” or “POM” includes metal-oxide or metal-oxygen ions (e.g., anions), clusters or cages in their various forms, including metal oxide cluster anions. In various embodiments, the POM may be included in a film on the electrode surface. Alternatively, the POM may be included as a doping ion in a polymer matrix to make an electrically active polymer. In addition, the POM may help to increase the charge storage capacity of the implantable electrode in which they are used due to POM redox properties (e.g., POM provides electroactive species with several oxidation states that allow for Faradaic redox transitions at the electrode/tissue interface). The pseudo-capacitance property of the POM can include a combination of porosity, the electro-active area (double layer) and Faradaic redox stages that POMs can go through. As used herein, a “film” refers to a layer of an electrically conductive substance which is deposited, directly and/or indirectly, on a surface of an implantable electrode.

Method embodiments for the present disclosure also include incorporating the POM into a polymerizable mixture and forming a film of the polymerizable mixture having the POM entrapped therein on the surface of the electrode. Examples of such methods include, but are not limited to, chemical or electrochemical generation of the polymer from a solution where the POM is present. The film formed during the electrochemical polymerization may include homogeneously entrapping the POM in the film.

Other deposition techniques are also possible. For example, the film that includes POM may be introduced into the film by an acid-base doping process after the film is formed. As will be appreciated, other processes may also be used to form the film, such as co-forming the film with the POM using a sol-gel process or other co-deposition process. Other deposition techniques include adsorption, self-assembly through electrostatic interactions, layer-by-layer deposition, and the Langmuir-Blodgett (LB) technique, among others.

The chemical composition and structures of the POM may also be adjusted according to various embodiments to alter electrical performance of the film on the surface of the electrode. For example, selection and use of the POM and additional doping anions incorporated in the film can be used to control the capacitance and impedance of the resulting implantable electrode. The electrode surface may further be porous to allow for an additional increase in effective surface area.

In addition to the POM acting as an electrical conductor, the film can also be formed of a conductive polymer that is doped with the POM. Examples of such conductive polymers include, but are not limited to, poly(pyrrole)s, poly(thiophene)s, polynaphthalenes, poly(acetylene)s, poly(aniline)s, poly(fluorene)s, polyphenylene, poly(p-phenylene sulfide), poly(para-phenylene vinylene)s, and polyfurane.

Embodiments of the implantable electrodes having POM may be suitable for use with wireless and wired electrodes. As used herein, an “electrode” includes an electrically conductive structure (e.g., an electrode body) that can be used to provide and/or sense an electrical potential to and from biological tissue. Examples of such electrodes include, but are not limited to, electrodes used for sensing and pacing cardiac tissue (e.g., pacing electrodes), sensing and delivering defibrillation energy to cardiac tissue (e.g., defibrillation electrodes), sensing electrical signals from and providing stimulation pulses to the nervous system including the brain, spinal cord, ear, and providing stimulation pulses to the vasculature system, to blood, and/or the urinary system. Such electrodes can have a coil configuration, a semi-hemispherical configuration, annular and/or semi-annular ring electrodes, all with or without active anchoring mechanisms (e.g., helical screw and/or tines).

In various embodiments, the electrode having the POM may be in the form of a lead having a lead body, a conductor in the lead body, and the electrode on the lead body having a surface that includes the POM. As discussed herein, the POM can be included in a film on the surface of the electrode. In an alternative embodiment, a wireless electrode may include a first and second electrode having a surface with the POM and an induction coil coupled between the first and the second electrode. The first and the second electrode may be used to produce an electrical potential discharge from energy (e.g., radio frequency energy) received with the induction coil. In addition, the wireless electrode can further include a battery coupled to the induction coil, where the battery may be rechargeable with current generated from the induction coil that receives radio frequency energy from an external transmitter. The wireless electrode may further include a storage capacitor coupled to the induction coil to store and deliver an electrical potential between the first electrode and the second electrode.

### BRIEF DESCRIPTION OF THE DRAWINGS

FIG. 1 illustrates an embodiment of a lead having an electrode, where the electrode has a film with a polyoxometalates (POM) according to the present disclosure.

FIG. 2 illustrates an embodiment of a wireless electrode with electrodes, where the electrodes have a film with POM according to the present disclosure.

FIG. 3 illustrates an additional embodiment of a wireless electrode with electrodes, where the electrodes have a film with POM according to the present disclosure.

### DETAILED DESCRIPTION

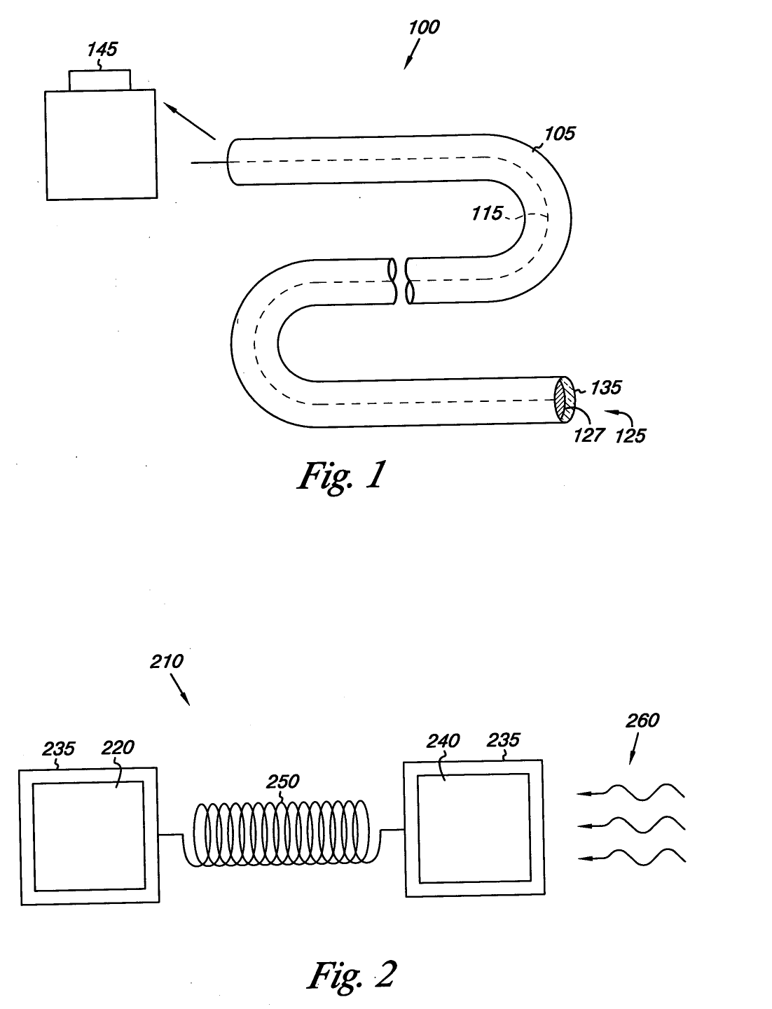

The Figures herein follow a numbering convention in which the first digit or digits correspond to the drawing Figure number and the remaining digits identify an element or component in the drawing. Similar elements or components between different Figures may be identified by the use of similar digits. For example, 110 may reference element “10” in FIG. 1, and a similar element may be referenced as 210 in FIG. 2. It should also be apparent that the scaling on the figures does not represent precise dimensions of the various elements illustrated therein.

The present disclosure provides for the incorporation of a metal oxide(s) into an electrode surface, thereby forming a nanocomposite structure. In particular, the present disclosure allows for polyoxometalates (POM), a class of metal oxide “clusters,” or compounds, to be incorporated into an electrode surface to allow for an increase in the electrochemically active and pseudo-capacitive surface area of the electrode without increasing the overall physical dimensions of the electrode.

POM displays a similarity in redox properties to pseudo-capacitive pacing electrodes such as iridium oxide (IrOx). Like IrOx, POM has the ability to undergo a reversible multi-electrode redox process. POM can also provide electroactive species with several oxidation states that allow for Faradaic redox transitions at an electrode/tissue interface. And like electrodes with IrOx, electrodes having POM may have lower polarization, higher capacitances, lower sensing impedance, and lower voltage thresholds.

According to the present disclosure, POMs may provide versatility in terms of structural, electrochemical, and photophysical properties of the resulting electrode surfaces. Electrode surfaces having POM incorporated therein help to reduce polarization losses of the electrode, while maintaining a satisfactory potential window for electrical stimulation delivered using the electrode. POM also displays good electrocatalytic activity in hydrogen peroxide and nitrogen oxide reductions which is beneficial for electrode applications. Electrode surfaces having the incorporated POM may also allow for charge transfer from the electrode without a significant loss of energy.

Generally, POM compounds recited in the present disclosure can be represented by the formula (I):

Aₐ[LₗMₘJ_{z}O_{y}] (I)

where A is at least one ion selected from the group consisting of Group 1-17 (IUPAC) elements, sodium (Na), potassium (K), ammonium, alkyl ammonium, alkyl phosphonium, and alkyl arsonium. L is at least one element selected from the group consisting of hydrogen and Group 13-17 elements. M is at least one metal selected from the group consisting of Group 4 and 7-12 metals. J is at least one metal selected from the group consisting of Group 5-6 metals. The subscript a is a number which when multiplied by the valence of A will balance the charge on the POM complex within the brackets. The subscript 1 is a number ranging from zero to about 20, the subscript m is a number ranging from zero to about 20, the subscript z is a number ranging from about 1 to about 50, and the subscript y is a number ranging from about 7 to about 150.

In one embodiment, L is at least one element of the group phosphorous (P), arsenic (As), silicon (Si), aluminum (Al), hydrogen (H), germanium (Ge), gallium (Ga), and boron (B); M is at least one element of the group zinc (Zn), titanium (Ti), manganese (Mn), iron (Fe), cobalt (Co), nickel (Ni), rhodium (Rh), zirconium (Zr), iridium (Ir), ruthenium (Ru), copper (Cu), and rhenium (Re); and J is at least one metal of the group molybdenum (Mo), tungsten (W), chromium (Cr), tantalum (Ta), and vanadium (V). In addition, subscript 1 ranges from zero to about 4; subscript m ranges from zero to about 6; subscript z ranges from about 6 to about 24; and subscript y ranges from about 18 to about 80.

Examples of POM compounds include, but are not limited to hexametalate anions [MₘJ₆₋ₘO_{y}], the Keggin anions [L₁ or ₂MₘJ₁₂₋ₘO_{y}], and the Dawson anions [L₂ to ₄MₘJ₁₈₋ₘO_{y}]. A specific example of a heteropolyoxometalate is the compound H₃PW₁₂O₄₀ which exhibits a typical molecular structure of a Keggin anion. Other examples of heteropolyoxometalates having the same structure include H₄SiW₁₂O₄₀, H₃PMo₁₂O₄₀, H₅PMo₁₀V₂O₄₀ and H₄PMo₁₀VO₄₀. It is understood that these examples are merely illustrative of heteropolyoxometalates and not intended to be limitative of the class of heteropolyoxometalates.

According to embodiments of the present disclosure, POM may be incorporated into the electrode surface. As discussed herein, this may be accomplished by forming a film that includes the POM on the electrode surface. The POM may then help to increase the electrochemically active surface area and the capacitance of existing conductive electrode materials without having to increase the size of the implantable device. The increase in active surface area and capacitance may even allow for a reduction in physical size of the implantable electrode, which would be beneficial in that it would promote ease of delivery and reduced tissue trauma. The use of POM in the electrode surface may also help to reduce polarization losses while remaining within a suitable potential window for electrical stimulation.

According to the present disclosure, a variety of immobilization techniques may be useful in incorporating POM with the electrode. For example, the POM may be bulk-entrapped in a polymer film that grows from a solution containing dissolved monomer and the POM during a chemical or electrochemical polymerization process. For example, during an electrochemical polymerization process, the monomer may be electrochemically oxidized at a polymerization potential giving rise to free radicals. These radicals can be adsorbed onto the electrode surface and subsequently undergo a wide variety of reactions leading to the polymer network that, while forming, entraps the POM. As the polymerization occurs locally on the electrode surface the POM would be entrapped in close proximity to the electrode surface. This is particularly suitable for the coating of electrode surfaces.

Other polymerizable conditions are also possible. These can include adsorption of the POM to the polymer film, chemical deposition, layer-by-layer (LBL) self-assembly of the POM on the polymer film through electrostatic interactions. Other LBL deposition techniques could also be used in incorporating the POM into the polymer film. In addition, sol-gel processing could be used to form films containing POM on electrodes. The Langmuir-Blodgett (LB) technique could also be used to form films (e.g., lamellar films) of the POM on the polymer film.

Control over the composition, structure, thickness, functional properties and orientation of a film that includes the POM can be influenced by the deposition technique and the conditions under which the film is produced. For example, the growth of a polymer film that includes the POM may depend on the electrical character of the polymer. In addition, polymer film generated by cycling the potential (e.g. potentiodynamically) or by generating at a fixed potential (e.g. potentiostatically) may also allow for a more precise control of the film thickness and its growth.

As discussed herein, the POM may also be incorporated into an implantable electrode by forming films of conductive polymers doped with POM anions onto the electrode surface. As used herein, a conductive polymer may include an organic polymer semiconductor that includes a band structure that allows for electrical conductivity. Exemplary conductive polymers include, but are not limited to, poly(pyrrole)s, poly(thiophene)s, polynaphthalenes, poly(acetylene)s, poly(aniline)s (leuco-emeraldine-base, emeraldine-base, and pernigraniline-base forms), poly(fluorene)s, polyphenylene, poly(p-phenylene sulfide), poly(para-phenylene vinylene)s, polyfurane, and their derivatives. The film may, for example, be grown by electropolymerization.

Additional examples of conductive polymers and/or doping ions that may be used with POM include those of a biological nature, those that display supercapacitive properties, trans- and cis-polyacetylene, and/or polyvinyl sulfonate (doping ion). For example, one embodiment of electrochemical polymerization on a positive anode substrate is to mix solutions of pyrrole, sodium polyvinyl sulfonate, and potassium polyoxymetalate and apply a potential of 0.4 volts (V) to 1.2 V to the anode. The desired doping level of the potassium POM anions may then be adjusted with the polymeric dopant of sodium polyvinyl sulfonate and/or polystyrene sulfonate. In one embodiment the POM is an isopoly anion of the form [MₘO_{y}]^{p-} or a heteropoly anion of the form [MₘJ_{z}O_{z}]^{q-} where M and J are as described herein.

Films of conductive polymers may also be formed by a layer-by-layer (LBL) self-assembly process which enables a layer-by-layer growth of films and the control of the composition, thickness, and orientation of each layer at the molecular level. As discussed, the LBL assembly process includes alternate adsorption of oppositely charged species via electrostatic attraction that can produce thin multilayer film structures. Also, the LBL self-assembly process can be used with POMs and diazoresin. In this case, the POM complexes with the diazopolymer were the usual ionic bonds formed between the compounds may be switched into covalent bonds, making a very stable thin film useful for long term applications in the body.

By way of example, multilayer films that include POM can by formed by the LBL process generally through a series of coating steps in aqueous solutions. During the coating steps, an electrode substrate can be dipped into a cationic aqueous solution containing a conductive polymer (e.g., polyaniline) and then into an anionic aqueous solution containing a POM. Molar concentrations of the solutions can be small (e.g. 0.1M, 0.01M or 0.001M) with an acidic pH (e.g., less than about pH=5). Such multilayer films can be formed by alternately immersing the desired electrode surface into the solutions of the cationic conductive polymer and the anionic POM for a predetermined time with intermediate water washing and drying.

In a further embodiment, POM may be incorporated at the electrode surface after polymerization of the film by acid-base doping. For example, the electrode surface can be made basic by the physical adsorption of a base, or chemical modification of the electrode surface with a base. A POM anion can then be introduced to the basic activated electrode surface to react with the base so as to form an adsorbed ion pair comprising POM anion and the protonated base. There may also be direct coordination by a donor atom to a peripheral heteroatom in a POM compound that possesses an open site or a weakly bound exchangeable ligand.

In additional embodiments, the concentration of POM anions in the electrode surface can be adjusted by co-incorporation of other doping anions. Other doping anions can be selected from the group consisting of biomolecules, including, but not limited to, tripolyphosphate, citrate, cyanate groups, heparin, or sulphate groups, for example. Use of the additional doping anions with the POM anions can allow for the electrode capacitance and impedance to be controlled and tailored by varying the chemical composition and doping level of the POM anions.

The electrode surfaces of the present disclosure can also have different physical configurations. For example, the electrode surfaces for receiving the conductive film can be porous, sintered, and/or patterned. Examples of suitable porous electrode surfaces include those materials selected from the group of platinum (Pt) and conductive ceramics such as iridium oxide, tungsten carbide, silicone carbide, titanium oxide-iridium oxide (TiO₂—IrO₂), iridium oxide-tantalum dioxide (IrO₂—TaO₂), tin oxide, indium oxide, and fullerene. These materials can be made porous by sputtering, electrodeposition, or sol-gel processes. On the other hand, the porous electrode surfaces can also be co-formed with POM anions using a process selected from the group of sol-gel processes, various methods of co-depositing (layer-by-layer self-assembly), and reactions with pendant surface ligands.

Additional electrode surfaces useful with the present disclosure include, but are not limited to, activated carbon, carbon aerogels, carbon foams derived from polymers, oxides, hydrous oxides, nitride ceramics such as TiN, carbides, nitrides and other conducting polymers. Examples of oxides and hydrous oxides include RuO₂, IrO₂, NiO, MnO₂, VOₓ, PbO₂ and Ag₂O. Also, examples of carbides and nitrides include MoCₓ, MO₂N, WCₓ and WNₓ.

As discussed herein, immobilized POM anions in the electrode surface can increase the number of conductive surface sites and the capacitance of the resulting electrode. For example, by adjusting the chemical composition of the POM anions structure (e.g., various combinations of ternary and binary mixed oxide combinations) the capacitance, polarization, electrochemical performance, and stability of the resulting electrode can be modified. Also, providing a larger surface area for the electrode through the use of the POM anions as described herein can decrease the current density and increase capacitance, all while the geometric surface area of the electrode remains substantially unchanged. Besides providing for a larger surface area, POM can also provide a combination of porosity, the electro-active area (double layer) and Faradaic redox stages that POMs can go through, as discussed herein.

Examples of such electrodes include, but are not limited to, electrodes used for sensing and pacing cardiac tissue, sensing and delivering defibrillation energy to cardiac tissue, sensing electrical signals from and/or providing stimulation pulses to the cells of the nervous and neurological system including the brain, spinal cord, ear, and providing stimulation pulses to the vasculature system, to blood, and/or the urinary system.

Embodiments of the electrode surfaces having the POM can be used with lead electrodes and/or with wireless electrodes. In various embodiments, the lead electrodes having the POM include a lead body, a conductor in the lead body, and an electrode on the lead body having a surface with the POM. In an alternative embodiment, the wireless electrode has a first and second electrode having a surface with the POM and an induction coil coupled between the first and the second electrode. The first and the second electrode can produce an electrical potential discharge from radio frequency energy received with the induction coil. In addition, the wireless electrode can further include a battery coupled to the induction coil, where the battery is rechargeable with current generated from the induction coil that receives radio frequency energy from an external transmitter. The wireless electrode can further include a storage capacitor coupled to the induction coil to store and deliver an electrical potential between the first electrode and the second electrode.

FIG. 1 provides an illustration of a lead 100. As shown, the lead 100 includes a lead body 105 with a conductor 115 in the lead body 105. The conductor 115 is shown coupled to an electrode 125 having surface 127. A pulse generator (e.g., a pacemaker) 145 is also shown, where the lead 100 can be releasably attached to the pulse generator 145 via a header structure. In one embodiment, the pulse generator 145 can include electronic components to perform signal analysis, processing and control. Such electronic components can include one or more microprocessors to provide processing and evaluation of sensed cardiac signals to determine and control delivery of electrical shocks and/or pulses of different energy levels and timing for ventricular fibrillation, atrial fibrillation, cardioversion, and/or pacing (dual or single chamber) to the heart in response to cardiac arrhythmias including fibrillation, tachycardia and bradycardia. The pulse generator 145 can also include a power supply, such a battery, a capacitor(s), and other components.

According to the present disclosure, the surface 127 of electrode 125 includes a film 135 having the POM formed according the embodiments of the present disclosure. Examples of materials for the electrode 125 are also according the embodiments of the present disclosure discussed herein. For example, material for the electrode 125 can include, but is not limited to, platinum (Pt), gold (Au), and iridium (Ir).

In an additional embodiment, the conductor 115 in the lead body 105 can also be formed, at least partially, from a polymer doped with the POM anions according to the present disclosure. For this embodiment, the polymer doped with the POM anions can be deposited, cast or extruded to form the conductor 115. In addition, it would be possible to co-extrude the POM anions doped polymer forming the conductor 115 with the surrounding lead body 105. Material selection for the lead body 105 can be from materials known in the art.

In a further embodiment, the lead 100 can be configured to be biodegradable. For example, the conductor 115 can be formed from deposited layers of POM around which is formed a lead body 105 of a biodegradable polymer. One way to form the biodegradable conductor 115 is to use the LBL self-assembly approach, creating layers of anionic POM with any suitable cationic counter molecule. For example, chitosan layers incorporated with POM can form ionic bonds between the layers, which can be slowly eroded by various salt ions in the body. Further examples of biodegradable polymers can include, but are not limited to, polycarboxylic acid, polyanhydrides including maleic anhydride polymers; polyorthoesters; poly-amino acids; polyethylene oxide; polyphosphazenes; polyactic acid, polyglycolic acid and copolymers and copolymers and mixtures thereof such as poly(L-lactic acid) (PLLA), poly (D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocaronates, and polydimethyl-trimethylcarbonates; cyanoacrylate; calcium phosphates; polyglycosaminoglycans; macromolecules such as polysaccharides (including hyaluronic acid, cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), and maleic anhydride copolymers.

In addition, the electrode 125, including the film 135 can be also be formed of a biodegradable conductive polymer doped with POM anions formed thereon according to the present disclosure. In this embodiment, the electrode 125 can be formed of a material prone to oxidation, such as iron (Fe) and/or magnesium (Mg).

FIG. 2 provides an illustration of a wireless electrode 210 according to the present disclosure. The wireless electrode 210 includes a first electrode 220 and a second electrode 240, with an induction coil 250 coupled between the electrodes 220, 240. One or both of the surfaces of the first and second electrodes 220, 240 can further include the film 235 having the POM according to the present disclosure. The induction coil 250 receives energy 260 that intersects the induction coil 250 at a parallel angle to produce an electrical potential discharge between the electrodes 220, 240.

In yet another embodiment, the wireless electrode 210 can be configured to be biodegradable. For example, the induction coil 230 can be made by building up layers of POM then insulating the POM with a biodegradable polymer insulator sheath. In addition, the electrodes 220 and 240 can be formed from one or more biodegradable polymers and/or the oxidizing metals, as discussed herein.

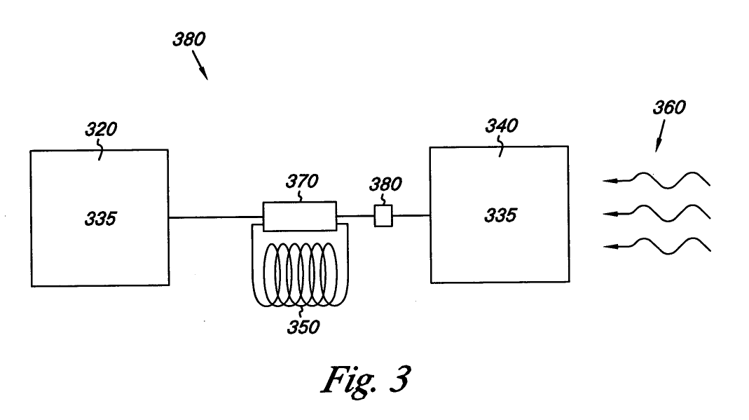

FIG. 3 provides an additional embodiment of the wireless electrode 310 that further includes a battery 370 and a storage capacitor 380 coupled to the induction coil 350 as well as an AC/DC converter (not shown). The battery 370 is rechargeable with current generated from the induction coil 250 from received RF energy 360 from an external transmitter. The storage capacitor 380 coupled to the induction coil 350 can then be used to store and deliver an electrical potential between the first electrode 320 and the second electrode 340. Examples of such wireless electrodes are provided in a commonly assigned U.S. patent application entitled “Leadless Cardiac Stimulation System” (BSCI Docket #04-0229), which is incorporated herein by reference in its entirety.

An additional embodiment of the present disclosure is to provide electrical stimulation to the surface of an implanted medical device having the POM anions to enhance healing of the surrounding tissues. For example, electrode surfaces having the POM anions as discussed herein can be integrated into surfaces of implants such as vascular grafts, synthetic heart valves, and left ventricular assist device (LVAD) surfaces where stimulation pulses are delivered to tissues adjacent the implant by an implanted or remote energy source. The voltage amplitude of the pulses must be adequate to stimulate cells, yet be below the threshold for noxious reactions at the electrode surface. This may be achieved in part by applying the films containing the POM to the electrodes that increase the electrode surface area without increasing the geometric surface area of the implant as described in the embodiments herein. Examples of such medical devices are provided in a commonly assigned U.S. patent application entitled “Stimulation of Cell Growth at Implant Surfaces ” (BSCI Docket #04-0062), which is incorporated herein by reference in its entirety.

The invention has been described with reference to various specific embodiments and described by reference to examples. It is understood, however, that there are many extensions, variations, and modification on the basic theme of the present invention beyond that shown in the examples and detailed description, which are within the spirit and scope of the present invention.

The complete disclosures of the patents, patent documents, and publications cited herein are incorporated by reference in their entirety as if each were individually incorporated. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

In the foregoing Detailed Description, various features are grouped together in several embodiments for the purpose of streamlining the disclosure. This method of disclosure is not to be interpreted as reflecting an intention that the embodiments of the disclosure require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. An implantable electrode comprising
an electrode surface having a polyoxometalate (POM).

2. The implantable electrode of claim 1, where the POM is included in a film on the electrode surface.

3. The implantable electrode of claim 2, where the film is a conductive polymer doped with the POM.

4. The implantable electrode of claim 3, where the conductive polymer is polypyrrole, polyvinyl sulfonate, polythiophene, polyaniline, or polyfurane.

5. The implantable electrode of claim 2, where the POM is entrapped in the electrode surface during an electropolymerization of the film on the electrode surface.

6. The implantable electrode of claim 2, where the film includes POM and a diazopolymer to provide the film.

7. The implantable electrode of claim 1, where the electrode surface provides for pseudo-capacitance electrode surface of the implantable electrode.

8. The implantable electrode of claim 1, where the electrode surface has a coating of a porous support.

9. The implantable electrode of claim 8, where the POM are co-formed with the porous support before the electrode is coated with the porous support.

10. The implantable electrode of claim 8, where the porous support is selected from the group consisting of platinum (Pt), iridium oxide, tungsten carbide, silicone carbide, titanium oxide-iridium oxide (TiO₂—IrO₂), iridium oxide-tantalum dioxide (IrO₂—TaO₂), tin oxide or indium oxide, and fullerene.

11. A wireless implantable electrode, comprising:
a first electrode having a surface with a polyoxometalate (POM);
a second electrode having a surface with a POM; and
an induction coil coupled between the first and the second electrode, where the first and the second electrode can produce an electrical potential discharge from radio frequency energy received with the induction coil.

12. The wireless implantable electrode of claim 11, where the POM is included in a conductive polymer film on the electrode surface.

13. The wireless implantable electrode of claim 11, where the wireless electrode is biodegradable.

14. The wireless implantable electrode of claim 11, including a battery coupled to the induction coil, where the battery is rechargeable with current generated from the induction coil that receives radio frequency energy from an external transmitter.

15. The wireless implantable electrode of claim 11, including a storage capacitor coupled to the induction coil to store and deliver an electrical potential between the first electrode and the second electrode.

16. A method, comprising:
incorporating a polyoxometalate (POM) into a polymerizable mixture; and
forming a film of the polymerizable mixture having the POM entrapped therein on a surface of an implantable electrode.

17. The method of claim 16, where forming the film includes performing an electropolymerization to form the film on the surface of the implantable electrode.

18. The method of claim 16, where the polymerizable mixture of the film is an electrically conductive polymer.

19. The method of claim 16, where forming the film includes homogeneously entrapping the POM in the film.

20. The method of claim 16, where forming the film includes a layer-by-layer process in which the POM is stabilized by polycations on the surface of the electrode.

21. The method of claim 16, including adjusting the chemical composition and structure of the POM to alter electrical performance of the film.

22. The method of claim 16, where the surface of the electrode has a porous structure formed by depositing a conductive material to form the electrode.
